(19) United States
(12) Patent Application Publication
(10) Pub. No.: US 2019/0064297 A1
(43) Pub. Date: Feb. 28, 2019

(54) METHOD OF PERFORMING MAGNETIC RESONANCE IMAGING AND A MAGNETIC RESONANCE APPARATUS

(71) Applicants: Siemens Healthcare Limited, Camberley (GB); King's College London, London (GB)

(72) Inventors: Radhouene NEJI, London (GB); Sebastien ROUJOL, Kingston upon Thames (GB)

(21) Appl. No.: 16/108,477

(22) Filed: Aug. 22, 2018

(30) Foreign Application Priority Data
Aug. 22, 2017 (GB) 1713443.8

(51) Int. Cl. G01R 33/56; A61B 5/055; G01R 33/28; G01R 33/48

(52) U.S. Cl. CPC G01R 33/5601; A61B 5/055; G01R 33/4828; G01R 33/5607; G01R 33/5608; G01R 33/286

(57) ABSTRACT

In a method of performing magnetic resonance imaging and a magnetic resonance apparatus, a region of interest in a subject in which a material having magnetic susceptibility has been introduced is imaged. A first imaging sequence includes excitation pulses having a frequency that is on-resonance is generated for application to the subject. A second imaging sequence includes excitation pulses having a frequency that is off-resonance is generated for application to the subject. Both the first and second imaging sequences have balanced gradient pulse trains. Signals emitted from the region of the interest in the subject in response to the first and second imaging sequences are detected, and first and second images are generated based on these signals. The first and second images are processed to generate a difference image.

### BACKGROUND OF THE INVENTION

### Field of the Invention

The present invention relates to a method of performing magnetic resonance imaging and a magnetic resonance apparatus. In particular, the present invention relates to a method of performing magnetic resonance imaging and a magnetic resonance apparatus for imaging a region of interest in a subject in which a material having a magnetic susceptibility has been introduced.

### Description of the Prior Art

Magnetic resonance (MR) imaging may be used to provide guidance for catheter-based interventions. Two different approaches are commonly used: passive tracking and active tracking.

MR-guided passive tracking is for visualizing a device within MR images based on the negative or positive contrast generated by intrinsic material characteristics of the device (e.g. its magnetic susceptibility). The contrast can be created and enhanced by incorporating ferromagnetic or paramagnetic materials into the device, or by using contrast agents. Specific imaging sequences have also been proposed to improve the visualization. MR guided active tracking uses active devices with embedded RF coils, antennas, or other sensors, to generate signals for localization.

The present disclosure is focused on passive catheter tracking in an MR environment.

One existing approach for passive catheter tracking is to use air or gadolinium filled balloons which provide negative or positive contrast visualization of the catheter.

Another existing approach for passive catheter tracking is to use a rigid guidewire or other metallic device such as a stent. These metallic devices allow for the creation of magnetic susceptibility artifacts in the MRI images which enable the guidewire/device to be visualized.

Another existing approach for passive catheter tracking is to use a partial slice refocusing gradient with a dual echo balanced steady-state free precession (bSSFP) sequence. This approach has been found to enhance device visualization by reducing the signal from surrounding tissues due to incomplete slice selection gradient refocusing. Complete slice selection gradient refocusing is achieved near the catheter due to the local magnetic susceptibility.

Another existing approach for passive catheter tracking is to use a low flip angle bSSFP sequence with slice gradient dephasing which enables positive contrast visualization of the catheter and the suppression of background signals.

For passive catheter tracking, it is desirable that the catheter be imaged with positive contrast, i.e. the catheter is seen in the MR images with hypersignal; both catheter and surrounding tissue should be visualized simultaneously for improved catheter guidance; and the imaging procedure should be fast enough to allow for a high temporal resolution for near real-time passive catheter tracking

### SUMMARY OF THE INVENTION

It is an object of the present disclosure to improve on the existing passive catheter tracking approaches, or at least provide an alternative to the existing passive catheter tracking approaches.

According to a first aspect of the invention, a method of performing magnetic resonance (MR) imaging on a region of interest in a subject in which a material having magnetic susceptibility has been introduced, comprising: includes a first imaging sequence for application to the subject, the first imaging sequence comprising excitation pulses having a frequency that is on-resonance and balanced gradient pulse trains, detecting first signals emitted from the region of interest in the subject in response to the first imaging sequence, and generating a first image based on the first signals; generating a second imaging sequence for application to the subject, the second imaging sequence comprising excitation pulses having a frequency that is off-resonance and balanced gradient pulse trains, detecting second signals emitted from the region of interest in the subject in response to the second imaging sequence, and generating a second image based on the second signals; and processing the first and second images to generate a difference image.

Here, an excitation pulse having a frequency that is “on-resonance” may mean that the excitation pulse has a frequency capable of inducing resonance in a spin or spin isochromat in the subject. The spin or spin isochromat may be subject tissue in the region of interest of the subject. As a result MR signals may be produced for the spin isochromat. The on-resonance frequency may be the same as or similar to the resonant frequency of the spin isochromat in the subject. The on-resonance frequency may be the Larmor frequency of the spin isochromat. A spin isochromat refers to a group of spins that resonate at the same frequency.

Here, an excitation pulse having a frequency that is “off-resonance” may have a frequency that is not capable of inducing resonance in the spin or spin isochromats in the subject. The spin or spin isochromat may be subject tissue in the region of interest of the subject. As a result MR signals may not be produced for the spin isochromat, or the intensity of the signals produced for the spin isochromat may be reduced as compared to the on-resonance frequency.

Since theoretically perfect operation of any MR apparatus is unlikely, it should be understood that the term “balanced” as used herein is meant to encompass substantially-preserved longitudinal and transverse magnetization, in which the net gradient-induced dephasing over a repetition time is substantially zero. Here, repetition time (TR) refers to the time between successive excitation pulses.

During the first imaging sequence, the on-resonance excitation pulses may result in tissue in the region of interest producing signals, but the material having magnetic susceptibility may not produce sufficient signals for visualization. This means that the tissue is in the passing band, but the material having magnetic susceptibility is a banding artifact and not clearly visible in the image. As a result, the first image may only be useful for visualizing background tissue, and not for visualization of the material having magnetic susceptibility. Significantly, during the second imaging sequence, the off-resonance excitation pulses may result in the signals produced by the material having magnetic susceptibility being increased, while the background tissue is reduced. This means that tissue in the region of interest may be a banding artifact and not clearly visible in the image, while the material having magnetic susceptibility is in the passing band.

Advantageously, by processing the first and second images to generate a difference image, the present disclosure results in a difference image where the magnetic susceptibility effects due to the material having magnetic susceptibility are enhanced, while the surrounding background tissue is reduced. The material having magnetic susceptibility may appear bright in the image, and thus has positive contrast. This means that the present disclosure enables the material having magnetic susceptibility to be imaged with positive contrast. The material having magnetic susceptibility may thus be clearly visualized in the difference image. In addition, the first image generated by the first imaging sequence may still enable the background tissue to be visualized with high signal-to-noise ratio.

Therefore, due to the susceptibility effects caused by the material having the magnetic susceptibility, the signal from the material is enhanced, and the signal from surrounding tissues is reduced. Furthermore, the acquisition of the first and second images may be fast enough to track the material with sufficiently high temporal resolution. The method of the first aspect does not require a guidewire or a contrast agent. In other words, the present invention uses dual on and off resonance excitation for passive tracking, allowing simultaneously the imaging of the material with positive contrast, signal reduction from the background tissue, and visualization of the anatomy.

The intensity of the signals produced the material having magnetic susceptibility may be reduced during the first imaging sequence and increased during the second imaging sequence. The on-resonance and off-resonance excitation pulses may be selected such that the signals produced by the material having magnetic susceptibility may be reduced during the first imaging sequence and increased during the second imaging sequence.

The material having magnetic susceptibility may cause local magnetic field distortion during the application of the first imaging sequence and the second imaging sequence. The local magnetic field distortion may reduce the intensity of signals produced by the material as a result of the application of the first imaging sequence, and may increases the intensity of signals produced by the material as a result of the application of the second imaging sequence.

The excitation pulses of the first imaging sequence may have a carrier frequency that is substantially the same as the resonant frequency of a spin isochromat in the subject, and wherein the excitation pulses of the second imaging sequence have a carrier frequency that is different from the resonant frequency of the spin isochromat. The resonant frequency of a spin isochromat, may mean the resonant frequency of tissue in the subject, such as tissue within the region of interest in the subject.

The excitation pulses of the second imaging sequence may have a carrier frequency shifted by approximately 1/(2×the repetition time (TR)) Hz with respect to the carrier frequency of the excitation pulses of the first imaging sequence. The specific frequency shift may vary from ½TR due to magnetic field inhomogeneity in the BO field. A frequency shift of ½TR may be used with good BO magnetic field homogeneity.

The phase of successive excitation pulses during the first imaging sequence may differ by a non-zero degrees phase increment. The phase of successive excitation pulses during the second imaging sequence may differ by zero degrees. In other words, the phase of successive excitation pulses during the second imaging sequence may not differ. The non-zero degrees phase increment may be 180 degrees.

The first imaging sequence and/or the second imaging sequence may be balanced steady state free precession (bSSFP) type sequences. bSSFP type sequences may have characteristic periodic bands, i.e. drops in signal, that have a periodicity of 1/TR Hz. Spins in the subject that have a resonant frequency in these periodic bands will not produce a signal, and thus will appear as banding artifacts in the resultant image. Significantly, by generating the second imaging sequence having off-resonance excitation pulses, this characteristic effect of bSSFP imaging is used to achieve surprising benefits. In particular, the spins that were previously on resonance (in the passing band) in the first imaging sequence now correspond to banding artifacts (drop in signal) in the second imaging sequence, and the regions that were exhibiting bSSFP banding artifacts in the first imaging sequence are now within the passing band (generate signal) in the second imaging sequence. Significantly, and advantageously, the present disclosure is able to exploit a characteristic of bSSFP to enhance the imaging of material having magnetic susceptibility.

The excitation pulses of the first imaging sequence may have a high flip angle. The high flip angle may be between 50-110 degrees. The high flip angle on-resonance bSSFP image allows for visualization of the anatomy with high SNR.

The excitation pulses of the second imaging sequence may have a high flip angle. The high flip angle for the second imaging sequence may be between 50-110 degrees. The high flip angle of the second imaging sequence may be the same as the high flip angle of the first image sequence. This helps prevent the introduction of contrast differences between the two images due to the flip angles.

The bSSFP type sequences may be single-shot bSSFP type sequences. The use of bSSFP type sequences, which may be single-shot, means that the material having magnetic susceptibility may be imaged sufficiently quickly for the material having magnetic susceptibility to be tracked with high temporal resolution.

Processing the first and second images to generate a difference image may result in a difference image in which the pixel values associated with the material having magnetic susceptibility are enhanced relative to the pixel values of background tissue in the subject.

Processing the first and second images to generate a difference image may comprise performing a subtraction operation on the first image and the second image. Performing a subtraction operation may mean that the first image is subtracted from the second image. This may mean that the pixel intensity values of pixels in the first image are subtracted from the pixel intensity values of corresponding located pixels in the second image. For example, if the pixel at location (x1, y1) in the first image has a pixel intensity value of 10 and the pixel at location (x1, y1) in the second image has a pixel intensity value of 5, then the difference image will have a pixel intensity value of −5 at location (x1, y1). Performing a subtraction operation may mean that the second image is subtracted from the first image.

The material having magnetic susceptibility may be a catheter.

The method may be performed in the absence of a guidewire and/or a contrast agent (such as carbon dioxide or Gadolinium). In other words, the method may enable the material having magnetic susceptibility to be imaged without requiring the use of a guidewire or a contrast agent.

The second imaging sequence may be generated after the first imaging sequence during an MR imaging procedure. The second imaging sequence may be generated shortly after the first imaging sequence. That is, the first imaging sequence and the second imaging sequence may be generated in quick succession.

The method of the first aspect may be used for tracking the movement of the material having the magnetic susceptibility in the region of interest in the subject during an MR interventional procedure.

According to a second aspect of the invention, a magnetic resonance (MR) apparatus for imaging a region of interest in a subject in which a material having magnetic susceptibility has been introduced, has a gradient system to apply a gradient magnetic field; an excitation system to apply an excitation pulse to the subject and to receive signals from the subject; and a computing system to receive the signals from the excitation system, the computing system to execute program code to: control the gradient system and the excitation system to generate a first imaging sequence for application to the subject, the first imaging sequence comprising excitation pulses having a frequency that is on-resonance and balanced gradient pulse trains, and to detect first signals emitted from the region of interest in the subject in response to the first imaging sequence; generate a first image based on the first signals; control the gradient system and excitation system to generate a second imaging sequence for application to the subject, the second imaging sequence comprising excitation pulses having a frequency that is off-resonance and balanced gradient pulse trains, and to detect second signals emitted from the region of interest in the subject in response to the second imaging sequence; and generate a second image based on the second signals; and to process the first and second images to generate a difference image.

The MR apparatus may be arranged to perform the method of the first aspect.

According to a third aspect, a non-transitory computer-readable medium is encoded with programming instructions (program code), when executed by a computer, computer system, or processor, cause the computer, computer system, or processor to perform the method of the first aspect.

### BRIEF DESCRIPTION OF THE DRAWINGS

FIG. 1 shows an example MR pulse sequence diagram for a first imaging sequence according to aspects of the invention.

FIG. 2 shows a detailed section of part of the MR pulse sequence diagram in FIG. 1.

FIG. 3 shows an example MR pulse sequence diagram for a second imaging sequence according to aspects of the invention.

FIG. 4 shows a detailed section of part of the MR pulse sequence diagram in FIG. 3.

FIG. 5a shows an example first image obtained using a first imaging sequence according to aspects of the invention.

FIG. 5b shows the first image in FIG. 5a with the colors inverted.

FIG. 5c shows an example second image obtained using a second imaging sequence according to aspects of the invention.

FIG. 5d shows the second image of FIG. 5c with the colors inverted.

FIG. 5e shows an example difference image obtained by processing the first and second images of FIGS. 5a and 5c.

FIG. 5f shows the difference image of FIG. 5e with the colors inverted.

FIG. 6 shows a process diagram for an example method according to the first aspect of the invention.

FIG. 7 shows an example MR apparatus according to the second aspect of the invention.

### DESCRIPTION OF THE PREFERRED EMBODIMENTS

The present invention relates to performing MR imaging on a region of interest in a subject in which a material having magnetic susceptibility has been introduced. The material having magnetic susceptibility in most examples is a catheter. In these examples, the present invention may enable the catheter to be tracked with positive contrast. This catheter tracking may be performed during an MR interventional procedure.

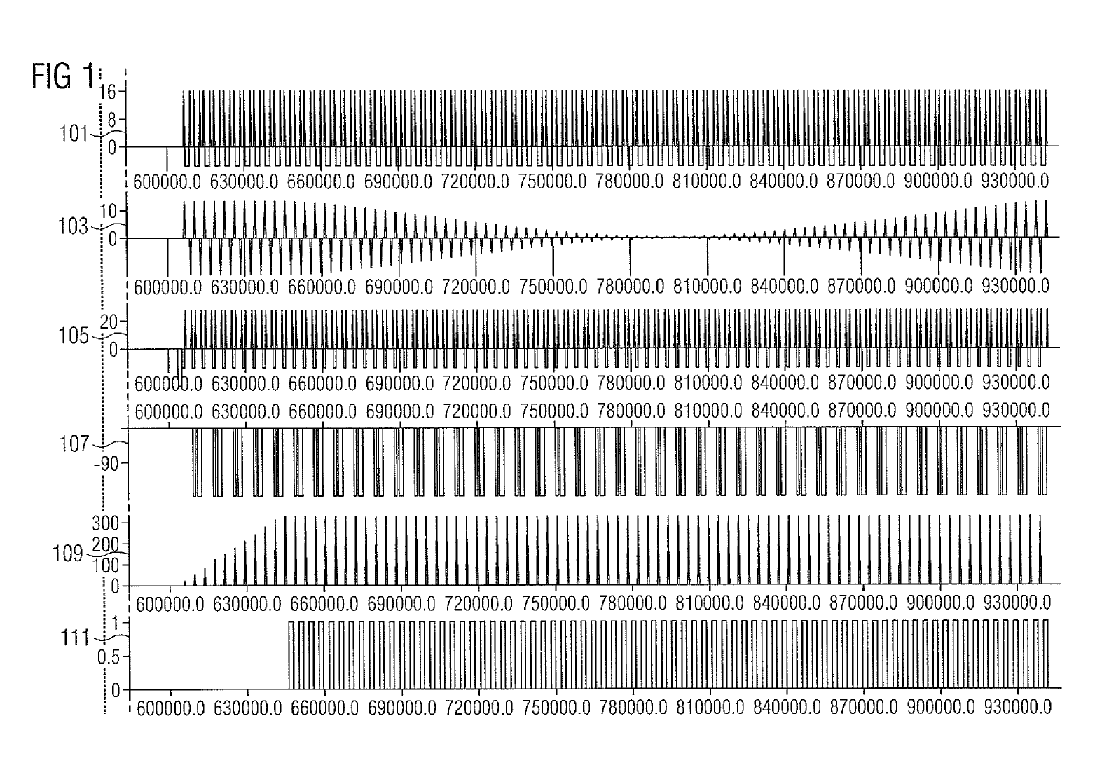

Referring to FIG. 1, there is shown an example MR pulse sequence diagram for a first imaging sequence according to aspects of the invention, The MR pulse sequence diagram shows the magnetic field gradients applied across the X 101, Y 103, and Z 105 axes so as to provide slice-select, frequency encode, and phase encode gradients. The magnetic field gradients 101, 103, 105 are balanced gradient pulse trains.

The first imaging sequence in this example is a high-flip angle, single-shot, balanced steady state free precession (bSSFP) type sequence. The use of bSSFP type sequence is advantageous as the present invention is able to exploit characteristic passing bands in the bSSFP signals that occur with periodicity 1/TR. TR is the repetition time and refers to the length of time between consecutive excitation pulses 109 in the first imaging sequence. In addition, the high flip angle on-resonance excitation pulses 109 mean that anatomy in the region of interest can be visualized with high signal-to-noise ratio. In this example, the high flip angle is between 50-110 degrees. Further, the single-shot sequence can be performed quickly enough to enable the catheter to be tracked during an MR interventional procedure.

The MR pulse sequence diagram further shows the excitation pulses 109 and the numeric crystal oscillator output 107. The numeric crystal oscillator is used to control the phase/frequency of the excitation pulses 109 applied to the subject as well as control the phase/frequency of the MR receiver used to receive signals from the subject.

The MR pulse sequence diagram further shows the analog-to-digital converter (ADC) output 111 which is activated during the acquisition of data after excitation pulses 109.

The excitation pulses 109 have a frequency that is on-resonance. In this example, this is achieved by increasing the phase of successive excitation pulses 109 by a non-zero degrees phase increment. In the particular example of FIG. 1, successive excitation pulses 109 have a 180 degrees phase increment.

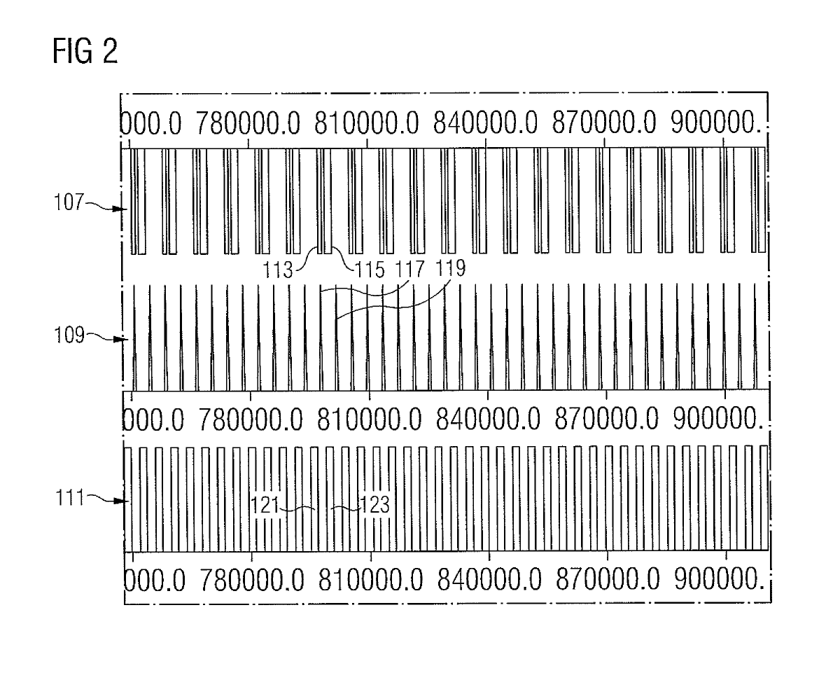

Referring to FIG. 2, there is shown a detailed section of part of the MR pulse sequence diagram shown in FIG. 1. In particular, a section of the numerical crystal oscillator output 107, excitation pulses 109 and analogue-to-digital converter (ADC) output 111 are shown in FIG. 2.

In the example of FIG. 2, the numeric crystal oscillator applies a −180 degrees phase shift 113 on a first excitation pulse 117. The numeric crystal oscillator then applies another −180 degrees phase shift 115 during the data acquisition stage such that the MR receiver has the same phase as the generated excitation pulse 117. The numeric crystal oscillator applies this −180 degrees phase shift 115 when the ADC is activated 121 to acquire first data. For the second excitation pulse 119 immediately following the first excitation pulse 117, the numeric crystal oscillator does not apply a phase shift. This means that the phase of the second excitation pulse 119 is increased by 180 degrees with respect to the first excitation pulse 117. The numeric crystal oscillator also does not apply a phase shift when the ADC is activated 123 to acquire second data after the generation of the second excitation pulse 119. It will be appreciated that this pattern repeats across successive excitation pulses 109 such that the phase is successively increased in increments of 180 degrees.

The first imaging sequence results in first signals being emitted from the region of interest in the subject. These first signals are detected and used to generate a first image.

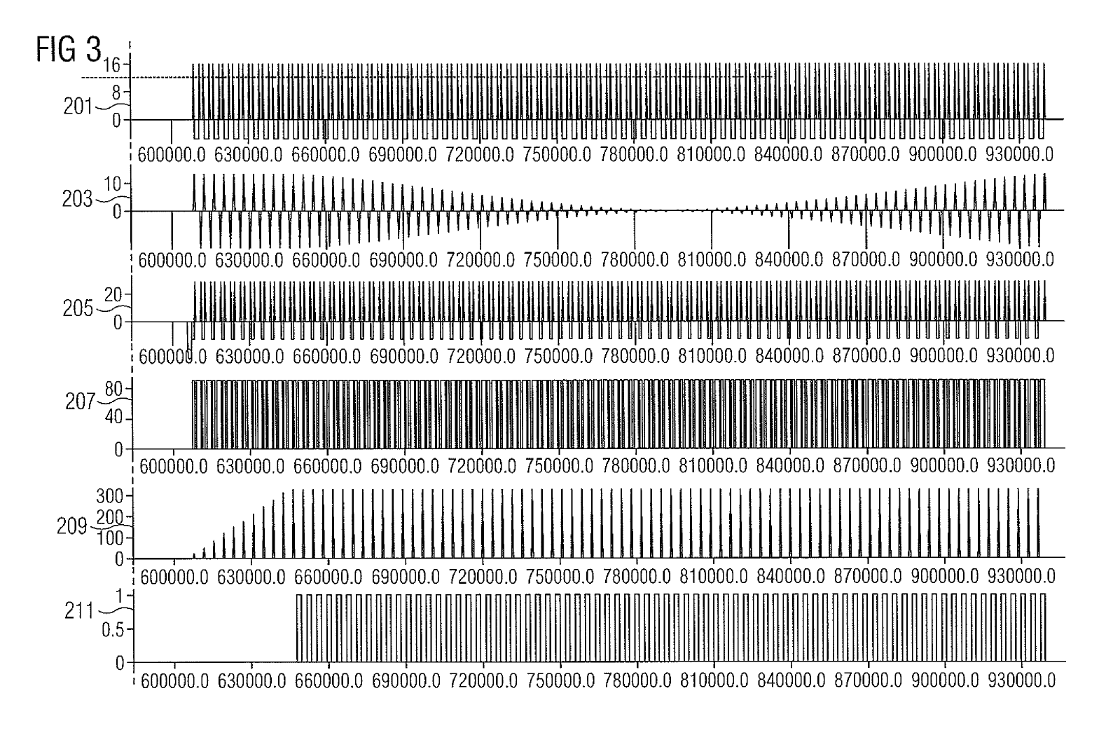

Referring to FIG. 3, there is shown an example MR pulse sequence diagram for a second imaging sequence according to aspects of the disclosure. The second imaging sequence is generated shortly after the first imaging sequence during an MR imaging procedure. The MR pulse sequence diagram shows the magnetic field gradients applied across the X 201, Y 203, and Z 205 axes so as to provide slice-select, frequency encode, and phase encode gradients. The magnetic field gradients 201, 203, 205 are balanced gradient pulse trains.

The second imaging sequence in this example is a single-shot balanced steady state free precession (bSSFP) type sequence. The second imaging sequence has the same flip angle as the first imaging sequence. The use of bSSFP type sequences is advantageous as the present disclosure is able to exploit characteristic passing bands in the bSSFP signals that occur with periodicity 1/TR. Further, the single-shot sequence can be performed quickly enough to enable the catheter to be tracked during an MR interventional procedure.

The MR pulse sequence diagram further shows the excitation pulses 209 and the numeric crystal oscillator output 207. The numeric crystal oscillator is used to control the phase/frequency of the excitation pulses 209 applied to the subject as well as control the phase/frequency of the MR receiver used to receive signals from the subject.

The MR pulse sequence diagram further shows the analogue-to-digital converter (ADC) output 211 which is during the acquisition of data after excitation pulses 209.

The excitation pulses 209 have a frequency that is off-resonance. In this example, this is achieved by not changing the phase of successive excitation pulses 209. In other words, the phase increment between successive excitation pulses 209 is 0 degrees.

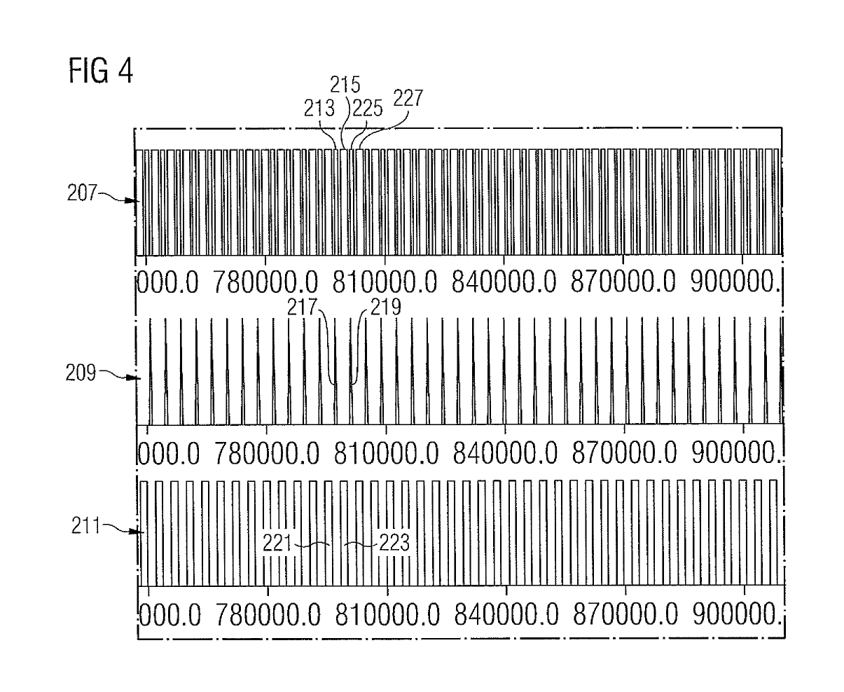

Referring to FIG. 4, there is shown a detailed section of part of the MR pulse sequence diagram shown in FIG. 3. In particular, a section of the numerical crystal oscillator output 207, excitation pulses 209 and analog-to-digital converter (ADC) output 211 are shown in FIG. 4.

In the example of FIG. 4, the numeric crystal oscillator applies a +90 degrees phase shift 213 on a first excitation pulse 217. The numeric crystal oscillator then applies another +90 degrees phase shift 215 during the data acquisition stage such that the receiver of the MR apparatus has the same phase as the generated excitation pulse 217. The numeric crystal oscillator applies this +90 degrees phase shift 215 when the ADC is activated 221 to acquire first data. For the second excitation pulse 219 immediately following the first excitation pulse 217, the numeric crystal oscillator again applies a +90 degrees phase shift 225. This means that the phase of the second excitation pulse 119 is not changed with respect to the first excitation pulse 217. The numeric crystal oscillator also applies a +90 degree phase shift 227 when the ADC is activated 223 to acquire second data after the generation of the second excitation pulse 219. It will be appreciated that this pattern repeats across successive excitation pulses 209 such that there is no phase shift across successive excitation pulses.

The second imaging sequence results in second signals being emitted from the region of interest in the subject. These second signals are detected and used to generate a second image.

While the above examples show generating the on-resonance excitation pulses 109 (FIG. 1) using a non-zero degree phase shifting scheme, and generating the off-resonance excitation pulses 209 (FIG. 3) using a zero degree phase shifting scheme, the present invention is not limited to this arrangement. In particular the same effect can be achieved by shifting the carrier frequency of the excitation pulses 209 of the second imaging sequence by approximately 1/(2TR) Hz as compared to the carrier frequency of the first imaging sequence. From an implementation point of view, the skilled person will appreciate that shifting the carrier frequency by ½TR Hz is equivalent to having a 0-degree phase cycling instead of the 180-degree phase-cycling.

In addition, it will be appreciated the present invention is not limited to the particular balanced gradient pulse trains 101, 103, 105, 201, 203, 205 as shown in FIGS. 1 and 3. It will be appreciated that other balanced gradient pulse trains can be selected as appropriate by those of ordinary skill in the MR technology, dependent on factors such as the MR apparatus and region to be imaged.

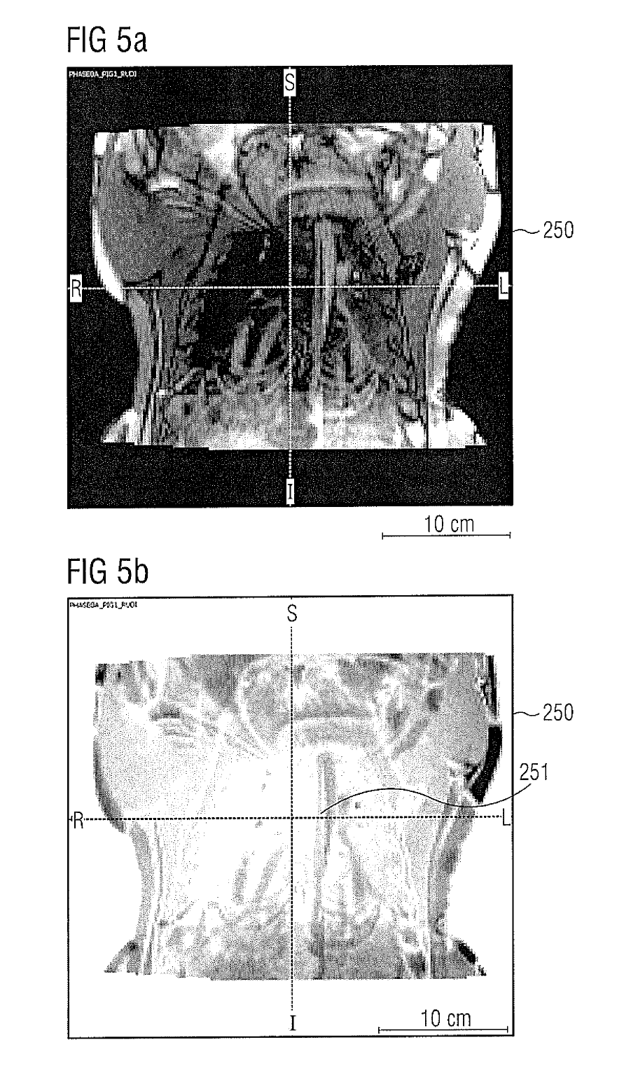

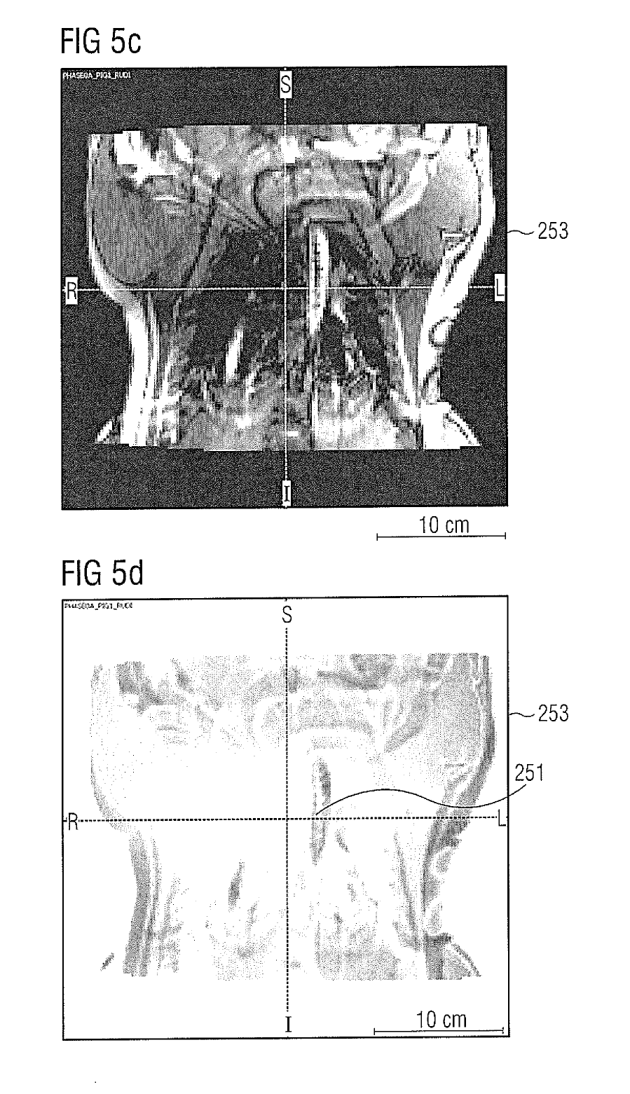

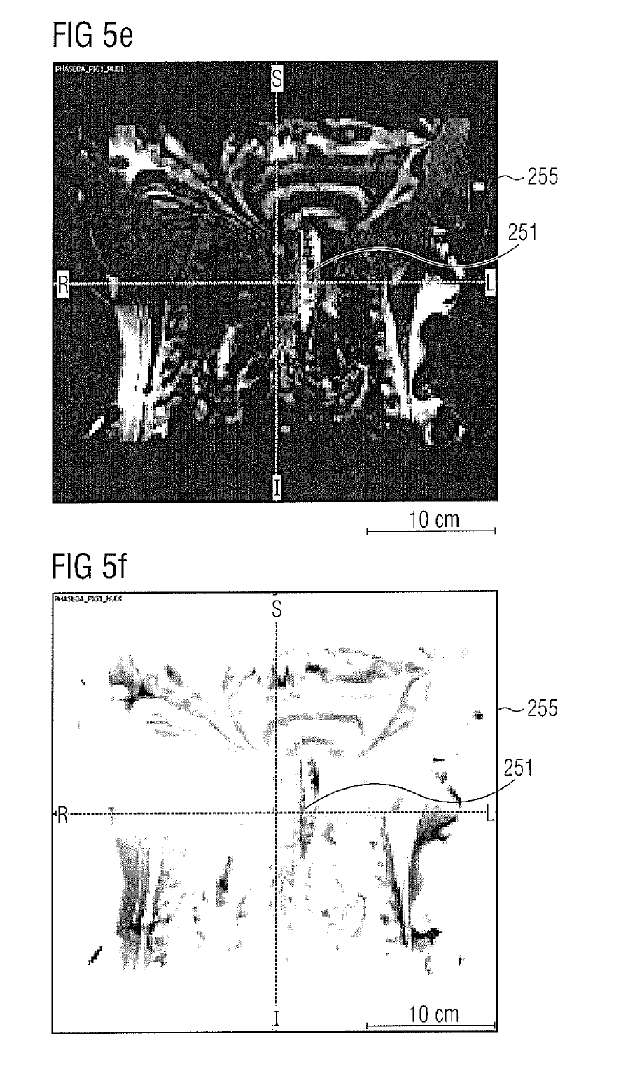

Referring to FIG. 5a, there is shown an example first image 250 of a region of interest in the subject generated as a result of a first imaging sequence. In this example first image 250, the anatomy of the subject is visible, but the presence of a catheter 251 is difficult to perceive. This is because the on-resonance excitation pulses 109 (FIG. 1) result in tissue in the region of interest producing signals, but the catheter 251 causes local magnetic field distortions which reduces the intensity of signals produced by the catheter 251. In other words, during the first imaging sequence, the catheter 251 corresponds to banding artifacts, that is a drop in signal, and the background tissue is within the passing band and generates a signal.

Referring to FIG. 5b there is shown the first image 250 of FIG. 5a but with the colors inverted for improved reproducibility. In FIG. 5b the location of the catheter 251 is indicated. It will be appreciated that the catheter 251 is not clearly separated/distinct from the background tissue of the first image 250. This means that it may be challenging for the medical professional to identify the presence/location of the catheter 251 with a high degree of confidence during an MR interventional procedure. In order to identify the presence/location of the catheter 251, the medical professional may have to carefully scrutinize the first image 250 which may take time, causing undesirable delays in the procedure.

Referring to FIG. 5c, there is shown an example second image 253 of a region of interest in the subject generated as a result of a second imaging sequence. In this example second image 253 the anatomy of the subject is visible but reduced as compared to the first image 250 of FIG. 5a. The signals generated in the region of the catheter 251 are enhanced as compared to the first image 250 of FIG. 5a. This is because the catheter 251 causes local magnetic field distortion that increases the intensity of signals produced by the catheter as a result of the application of the second imaging sequence with the off-resonance excitation pulses 209 (FIG. 3). In other words, during the second imaging sequence, the catheter 251 is within the passing band and generates a signal, while the background tissue corresponds to banding artifacts (a drop in signal).

Referring to FIG. 5d there is shown the second image 253 of FIG. 5c but with the colors inverted for improved reproducibility. In FIG. 5d the location of the catheter 251 is indicated. It will be appreciated that the catheter 251 is more visible than compared to the first image 250, but is still not clearly separated/distinct from the background tissue of the second image 253.

Referring to FIG. 5e, there is shown a difference image 255 obtained by processing the first image 250 and the second image 253. In particular, the difference image 255 is obtained by subtracting the first image 250 from the second image 253. The difference image 255 highlights the appearance of the catheter 251, and reduces the appearance of the background tissue. This means that the difference image 255 enables the medical professional to quickly and confidently identify the presence/location of the catheter 251 in the region of interest.

Referring to FIG. 5f there is shown the difference image 255 of FIG. 5e but with the colors inverted for improved reproducibility.

Significantly, processing the first and second images 250, 253 to generate a difference image 255, results in a difference image 255 where the magnetic susceptibility effects due to the catheter 251 are enhanced (with positive contrast), while the surrounding background tissue is reduced. This means that the difference image 255 enables the catheter 251 to be imaged with positive contrast. The first image 250 generated by the first imaging sequence still enables the background tissue to be visualized with high signal-to-noise ratio.

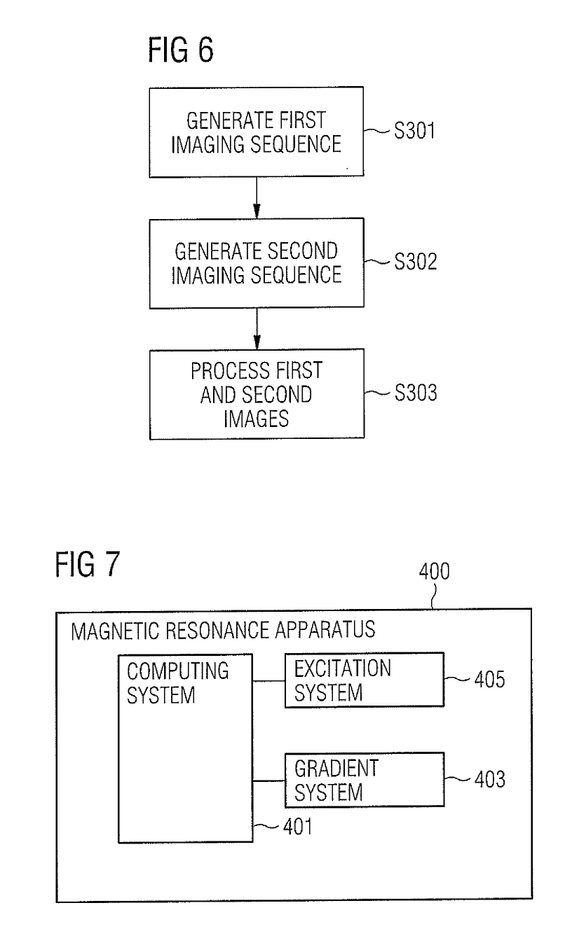

Referring to FIG. 6, there is shown an example method according to the first aspect of the invention.

Step 301 involves generating a first imaging sequence for application to the subject. The first imaging sequence comprises excitation pulses having a frequency that is on-resonance and balanced gradient pulse trains. Step 301 further involves detecting first signals emitted from the region of interest in the subject in response to the first imaging sequence, and generating a first image based on the first signals.

Step 302 involves generating a second imaging sequence for application to the subject. The second imaging sequence comprises excitation pulses having a frequency that is off-resonance and balanced gradient pulse trains. Step 302 further involves detecting second signals emitted from the region of interest in the subject in response to the second imaging sequence, and generating a second image based on the second signals.

The first and second imaging sequences generated during steps 301 and 302 may be the same as the example imaging sequences described above in relation to FIGS. 1 to 4. However, the present disclosure is not limited to these particular imaging sequences.

Step 303 involves processing the first and second images to generate a difference image.

Referring to FIG. 7, there is shown an example MR apparatus 400 according the second aspect of the disclosure. The MR apparatus 400 has a scanner with a gradient system 403, excitation system 405, and computing system 401. The gradient system 403 applies a gradient magnetic field. The excitation system 405 applies an excitation pulse to the subject and receives signals from the subject. The computing system 401 receives the signals from the excitation system 405.

The computing system 401 also executes program code to control the gradient system 403 and the excitation system 405, to generate a first imaging sequence for application to the subject, and to detect first signals emitted from the region of interest in the subject in response to the first imaging sequence. The computing system 401 also executes program code to generate a first image based on the first signals. The first imaging sequence includes excitation pulses having a frequency that is on-resonance and balanced gradient pulse trains.

The computing system 401 also executes program code to control the gradient system 403 and the excitation system 405, to generate a second imaging sequence for application to the subject, and to detect second signals emitted from a subject in response to the second imaging sequence. The second imaging sequence includes excitation pulses having a frequency that is off-resonance and balanced gradient pulse trains. The computing system 401 also executes program code to generate a second image based on the second signals.

The computing system 401 also executes program code to process the first and second images to generate a difference image.

The scanner of the MR apparatus 400 includes a magnet (not shown) for establishing a stationary magnetic field. The magnet can include a permanent magnet, a superconducting magnet or other type of magnet. The excitation system 405 includes a transmitter (not shown) and a receiver (not shown). The excitation system 405 can be an RF system with one or more RF coils (not shown). The gradient system 403 includes one or more coils (not shown) used to apply magnetic gradients for localization during MR imaging.

The computing system 401 is in communication with the gradient system 403 and excitation system 405 for controlling these components. The computing system 401 can include processing circuitry (not shown) configured to execute program code for controlling the MR apparatus 400 to perform the method of the first aspect. The computing system 401 could be an integrated component of the MR apparatus 400. The computing system 401 could be a desktop computer, a workstation, a server, or a laptop computer.

According to aspects of the invention, there is also provided a computer-readable medium having instructions recorded thereon which, when executed by a processing device, cause the processing device to perform the method of the first aspect.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements.

The described and illustrated embodiments are to be considered as illustrative and not restrictive in character, it being understood that only the preferred embodiments have been shown and described and that all changes and modifications that come within the scope of the inventions as defined in the claims are desired to be protected. It should be understood that while the use of words such as “preferable”, “preferably”, “preferred” or “more preferred” in the description suggest that a feature so described may be desirable, it may nevertheless not be . necessary and embodiments lacking such a feature may be contemplated as within the scope of the invention as defined in the appended claims. In relation to the claims, it is intended that when words such as “a,” “an,” “at least one,” or “at least one portion” are used to preface a feature there is no intention to limit the claim to only one such feature unless specifically stated to the contrary in the claim. When the language “at least a portion” and/or “a portion” is used the item can include a portion and/or the entire item unless specifically stated to the contrary.

In summary, there is provided a method of performing magnetic resonance (MR) imaging, an MR apparatus, and a computer readable medium. A region of interest in a subject in which a material having magnetic susceptibility has been introduced is imaged. A first imaging sequence comprising excitation pulses having a frequency that is on-resonance is generated for application to the subject. A second imaging sequence comprising excitation pulses having a frequency that is off-resonance is generated for application to the subject. Both the first and second imaging sequences have balanced gradient pulse trains (S301, S302). Signals emitted from the region of the interest in the subject in response to the first and second imaging sequences are detected, and first and second images are generated based on these signals. The first and second images are processed to generate a difference image (S303).

All of the embodiments and features herein, and/or all of the steps of any method or process disclosed herein, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

Although modifications and changes may be suggested by those skilled in the art, it is the intention of the Applicant to embody within the patent warranted hereon all changes and modifications as reasonably and properly come within the scope of the Applicant's contribution to the art.

1. A method of performing magnetic resonance (MR) imaging on a region of interest in a subject in which a material having magnetic susceptibility has been introduced, comprising:
generating a first imaging sequence for application to the subject, the first imaging sequence comprising excitation pulses having a frequency that is on-resonance and balanced gradient pulse trains, detecting first signals emitted from the region of interest in the subject in response to the first imaging sequence, and generating a first image based on the first signals;
generating a second imaging sequence for application to the subject, the second imaging sequence comprising excitation pulses having a frequency that is off-resonance and balanced gradient pulse trains, detecting second signals emitted from the region of interest in the subject in response to the second imaging sequence, and generating a second image based on the second signals; and
processing the first and second images to generate a difference image.

2. A method as claimed in claim 1, wherein the intensity of the signals produced by the material having magnetic susceptibility are reduced as a result of the first imaging sequence and increased as a result of the second imaging sequence.

3. A method as claimed in claim 2, wherein the material having magnetic susceptibility causes local magnetic field distortion during the application of the first imaging sequence and the second imaging sequence, wherein the local magnetic field distortion reduces the intensity of signals produced by the material during the application of the first imaging sequence, and increases the intensity of signals produced by the material during the application of the second imaging sequence.

4. A method as claimed in claim 1, wherein the excitation pulses of the first imaging sequence have a carrier frequency that is substantially the same as the resonant frequency of a spin isochromat in the region of interest, and wherein the excitation pulses of the second imaging sequence have a carrier frequency that is different from the resonant frequency of the spin isochromat.

5. A method as claimed in claim 1, wherein the excitation pulses of the second imaging sequence have a carrier frequency shifted by approximately 1/(2×the relaxation time) Hz with respect to the carrier frequency of the excitation pulses of the first imaging sequence.

6. A method as claimed in claim 1, wherein the phase of successive excitation pulses during the first imaging sequence differ by a non-zero degrees phase increment, and wherein the phase of successive excitation pulses during the second imaging sequence differ by zero degrees.

7. A method as claimed in claim 6, wherein the non-zero degrees phase increment is 180 degrees.

8. A method as claimed in claim 1, wherein processing the first and second images to generate a difference image comprises subtracting the first image from the second image.

9. A method as claimed in claim 1, wherein the material having magnetic susceptibility is a catheter.

10. A method as claimed in any claim 1, wherein the first and/or second imaging sequence are balanced steady-state free precession (bSSFP) type sequences.

11. A method as claimed in claim 1, wherein the bSSFP type sequences are single-shot bSSFP type sequences.

12. A method as claimed in claim 1, wherein the excitation pulses of the first imaging sequence and second imaging sequence have a flip angle of between 50-110 degrees.

13. A magnetic resonance (MR) apparatus for imaging a region of interest in a subject in which a material having magnetic susceptibility has been introduced, the apparatus comprising:
a gradient system to apply a gradient magnetic field;
an excitation system to apply an excitation pulse to the subject and to receive signals from the subject; and
a computing system that receives the signals from the excitation system, the computing system being configured to:
control the gradient system and the excitation system to generate a first imaging sequence for application to the subject, the first imaging sequence comprising excitation pulses having a frequency that is on-resonance and balanced gradient pulse trains, and to detect first signals emitted from the region of interest in the subject in response to the first imaging sequence;
generate a first image based on the first signals;
control the gradient system and excitation system to generate a second imaging sequence for application to the subject, the second imaging sequence comprising excitation pulses having a frequency that is off-resonance and balanced gradient pulse trains, and to detect second signals emitted from the region of interest in the subject in response to the second imaging sequence;
generate a second image based on the second signals; and
process the first and second images to generate a difference image.

14. A non-transitory, computer-readable data storage medium encoded with programming instructions, said storage medium being loaded into a computer system of a magnetic resonance (MR) apparatus comprising a gradient system and an excitation system, said programming instructions causing said computer system to:
operate the gradient system and the excitation system in order to generate a first imaging sequence applied to a subject, said first imaging sequence comprising excitation pulses having a frequency that is on-resonance and comprising balanced gradient pulse trains, and detect first signals in response to the first imaging sequence, emitted from a region of interest of the subject in which a material having magnetic susceptibility has been introduced;
generate a first image from said first signals;
operate the gradient system and the excitation system to generate a second imaging sequence applied to the subject, said second imaging sequence comprising excitation pulses having a frequency that is off-resonance and comprising balanced gradient pulse trains, and detect second signals emitted from said region of interest in response to the second imaging sequence;
generate a second image from the second signals; and
process the first and second images in order to generate a difference image.
